(19) United States
(12) Patent Application Publication
(10) Pub. No.: US 2006/0134040 A1
(43) Pub. Date: Jun. 22, 2006

(54) Pest protection method and composition

(76) Inventors: David Glassel, Spring, TX (US); Gary Erwest Mellema, Grapevine, TX (US)

Correspondence Address: Martin L. McGregor, 26415 Oak Ridge Drive, Spring, TX, 77380, US

(21) Appl. No.: 11/016,584

(22) Filed: Dec. 17, 2004

(51) Int. Cl. A61K 8/00

(52) U.S. Cl. 424/70.1

(57) ABSTRACT

A method and composition for the method for preventing insect infestation that comprises applying to the area to be protected an effective amount of solid material coated with by treatment with a composition comprising a silicone polymer, and an essential oil. A protective effect by reducing the population of harmful biological pests in the locality to be protected is provided.

### TECHNICAL FIELD

This invention provides methods and compositions of matter for protecting against biological pests. The methods and compositions of the invention are especially useful in protecting growing plants and buildings against insects by applying an essential oil coated on a solid carrier.

### BACKGROUND OF THE INVENTION

Repealing insects by treatment with various agents have been practiced for many years. The products normally used for repealing insects generally comprise one or more active substances that are toxic to at least some organisms.

This invention relates to insect, plant, or microbial control compositions and methods of using same to control various crawling and flying insect pests, and, in its preferred embodiments, relates more particularly to “non-poison” insecticides adapted to increase insect protection by repellent qualities as well as increasing mortality. Commercially available insecticides, including those available for home use, commonly comprise active ingredients or “poisons” which are not only toxic to the target insect pests, but, if used in relatively confined environments and delivered as aerosol sprays, can be present in sufficient concentration to also be toxic to humans and household pets. Various undesirable side effects may include immediate or delayed neurotoxic reactions, and/or suffocation. Even the noxious odor of such materials can cause headaches or upset stomachs in some individuals. These adverse side effects are exacerbated when such compositions come in contact with persons of increased sensitivity, or persons of small body mass such as children or babies. For some time, efforts have been made to develop insecticidal compositions, particularly those intended for residential use in aerosol form, which are effective in killing the targeted insect pests completely and quickly, but nontoxic to humans and pets. It has now been found to be even more desirable simply to deter insect infestation because high kill rates accelerate the development of immune insect populations. The Environmental Protection Agency (EPA) regulates the use of potentially toxic ingredients in pesticidal compositions under the Federal Insecticide, Fungicide and Rodenticide Act. Certain materials considered either active or inert materials by the EPA have been deregulated or otherwise identified as acceptable “safe” substances offering minimum risk in normal use. Other materials are currently undergoing investigation and may be deregulated in due course. Deregulated substances are generally considered non-poisonous by the consumer. Thus, the term “non-poisonous” as used herein is intended to convey a composition that, while highly effective in killing or repelling targeted insect pests, is safe to use around humans, particularly small children, and pets. Non-poisonous insecticidal compositions available heretofore incorporating deregulated materials as the active ingredient were believed to have had limited efficacy due to low kill rates, the benefits of repellents were vastly under rated. Attempts to use deregulated essential oils as the active ingredient in such insecticides, while having limited success, have generally been found to be either cost prohibitive, inadequately lethal to control a range of targeted insect pest species, or too slow-acting to enable the user to confirm that the insect has been killed and to dispose of the dead insect so as to avoid polluting the environment. Among the insects which are found to be particularly undesirable are cockroaches, both the American and German species. These pests shed their “skin” which, over time, disintegrates forming what is known as “cuticle” in the air, a particular problem for people suffering from asthma. Thus, not only is it important to kill cockroaches with an effective insecticide, the kill time must be sufficiently short for the carcass to be properly disposed of before the insect can crawl into a remote area to die. While cockroaches are a prime target for a household spray, for general application, such materials must also be effective against other crawling pests, such as ants, water bugs, silverfish, crickets, spiders and centipedes. Additionally, aerosol compositions of such insecticides of proper concentration must also be effective against various flying insects, including flies, mosquitoes, gnats, moths, wasps, hornets, yellow jackets, bees and other flying insects, both inside and outside of the house. Essential oils currently deregulated by the EPA include cedar oil, cinnamon oil, citronella oil, clove oil, corn oil, garlic oil, lemongrass oil, linseed oil, peppermint oil, rosemary oil, soybean oil and thyme oil. Among the essential oils proposed for exemption from registration are a number of the citrus oils. Citrus oils would include orange oil, lemon oil, lime oil, grapefruit oil and tangerine oil. Some of these other essential oils have been considered for their insecticidal or insect repellent properties, but they have not been shown to be particularly effective as active ingredients. In contrast, cedar oil, citronella oil, clove oil, peppermint oil and others have long been recognized as highly effective. Therefore, it would be both environmentally and commercially important to be able to enhance the properties of these relatively safe essential oils by a formulation and application method that provides a protective effect in a cost effective manner. It is also desirable to select the particular active ingredient to protect against the indigenous insect populations in a particular location. For many applications, it has been found to be especially desirable to combine an essential oil with a silicone polymer and a solid carrier. It is especially convenient to use locally available natural materials such as solid agricultural wastes, for example corn cob prills, as a carrier.

### SUMMARY OF THE INVENTION

The invention provides a composition of matter useful for preventing biological pest infestations that comprises a solid and a biologically effective amount of an oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil, or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil mixed with a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable and a crosslinking catalyst wherein the base copolymer is 75 to 90% by weight and the crosslinker is 9 to 24% by weight and the catalyst is 1 to 5% by weight of the component. The core may be any solid material, for example it may be a particulate clay, expanded clay, diatomaceous earth, glass beads, silica gel beads, zeolites, soil amendments, such as gypsum or calcium sulfate particles, lime, and the like, nontoxic spent catalyst waste; fertilizer, time release fertilizer pellets, grain casings such as, rice hulls, wheat chaff and the like, nut shells such as peanut hulls, pecan shells, almond shells, walnut shells, pistachio shells, hazelnut shells, and the like, wood particles such as pelletized wood dust, sawdust, wood chips, wood shavings bark mulch, vegetative matter such as processed soy bean meal, dried vegetable wastes, pelletized vegetative matter, coir, corn cob prills, mulch, and the like. Preferably, the foregoing mixture is applied as a coating to an absorbent plant material as a particulate carrier, the currently most preferred core material is corncob prill. The particle size may be any size that is convenient for application, however the more finely divided the material, the better it is expected to perform so long as it is not so finely divided and as to not settle when applied. Preferably, the size range is the same as that used for other solids applied to growing plants such as soil amendments, mulches or fertilizers. Preferably, the coating composition is from 1 to 99% preferably 70 to 90% essential oil, more preferably about 80% essential oil and the residue is substantially the silicone polymer. The composition preferably includes an oil selected from the group consisting of cedar oil, cinnamon oil, citronella oil, clove oil, eucalyptus oil, juniper oil, tall oil, and pine oil especially preferred is cedar oil, also known as cedar wood oil.

The invention may also be considered as a method of treatment useful for protecting an area against insect infestation that comprises applying an effective amount of a particulate solid material with a coating comprising a mixture of the following components: (1) at least a biologically effective amount of an oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and (2) at least 10% of a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and a crosslinking catalyst wherein the base copolymer is 75 to 90% by weight and the crosslinker is 9 to 24% by weight and the catalyst is 1 to 5% by weight of the component. Preferred natural oils include cedar oil, cinnamon oil, citronella oil, clove oil, eucalyptus oil, juniper oil, tall oil, and pine oil, cedar oil (also known as cedar wood oil) is especially preferred. Preferred carriers are materials that are normally applied to growing plants such as mulches, fertilizers, soil amendments, and the like, or materials that may be readily mixed with such materials such as a corncob prill.

The invention may also be considered as a composition useful in practicing the method of the invention that comprises a plant material coated with a mixture comprising the following components: (1) a biologically effective amount in the range or 1 to 99% by weight of a natural product oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and (2) the balance comprising a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and a crosslinking catalyst. Preferably, the mixture may also comprise one or more agricultural adjuvants such as surfactants and the like. Preferred compositions are those wherein the base copolymer is 75 to 90% by weight and the crosslinker is 9 to 24% by weight and the catalyst is 1 to 5% by weight of the component and the overall mixture is from 65 to 95% by weight aliphatic solvent, a biologically effective amount of an oil, preferably from 1 to 80% by weight oil and the silicone based polymer is at least 5% by weight. The composition also comprises a catalyst that promotes film formation in the silicone-based component. While any catalyst may be used preferred catalysts are metal soaps, especially preferred are crosslinking catalysts selected from the group consisting of metal salts of alkylcarboxylic acids having from 2 to 18 carbons, and more especially preferred metal soaps are tetraalkyl titanates or zirconates.

The invention may also be considered to be a method of preparing a biologically active plant protection composition comprising the steps of mixing a particulate solid material in a chamber and spraying the solid during the mixing of the solid with a mist of a composition that comprises (1) a quantity of a selected oil sufficient to provide a biologically effective amount in the finished product of at least one oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and the balance further comprising at least one silicone based polymer that forms a film in the presence of a catalyst and water comprising at least one component selected from the group consisting of (A) a copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; and (B) (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and (C) a crosslinking catalyst and curing the mixture in the presence of water to provide a free flowing particulate solid material.

### DETAILED DESCRIPTION OF THE INVENTION

### GENERAL DESCRIPTION OF THE INVENTION

### DEFINITIONS

A “biologically effective amount of an oil” means enough material is present to produce a statistically significant increase in protection against at least one biological pest over a control treated with substantially the same materials but for the biologically active oil. Unless otherwise noted all terms used herein have the meaning normally ascribed in the arts that use the terms in the normal course of practice in that art. A biological effect may be a lethal effect or a non-lethal effect such as repelling an insect or inhibiting feeding or inhibiting the growth of a pest plant or micro-organism or interrupting the life-cycle of a pest organism or deterring spore formation or attracting to the area or sustaining the growth of a beneficial insect or micro organism in the area to be protected.

### EXAMPLE 1

### PREPARATION OF COMPOSITIONS OF THE INVENTION

The compositions of the invention comprises a particulate core, preferably a material normally applied to growing plants, more preferably an absorbent plant material such as a mulch or a corn cob prill, the core being coated with an essential oil in a silicone polymer that is a mixture of alkylsiloxanes having a general base formula of: (MₐD_{b}T_{c}Q_{d})ₓ where M is R₃SiO_{1/2}—; D is R₂SiO—; T is RSiO_{3/2}—; and Q is Si(O_{1/2})₄— and R is a generalized organic radical selected from: linear or branched hydrocarbon radicals of 1-8 carbons containing 0-1 degree of unsaturation, or phenyl, or trifluoropropyl radicals, and may optionally be substituted with a hydroxyl, alkoxy or acyloxy group of 1 to 8 carbons.

A preferred embodiment is ^{HO}MDₓM^{OH} namely a silanol endblocked polydimethylsiloxane. The preferred viscosity is 50-3500 cSt with 750-1500 cSt being preferred. The composition is subject to the following general parameters:

The ratio of a/(c+d) is between 0 and 4 with the preferred range being 0-0.5.

The ratio of b to the rest is not subject to limitation provided the final base viscosity is between 50-3500 cSt with 750-1500 being preferred. R at each position may be the same or different and will be predominately methyl. All R groups being methyl is a preferred choice. In addition, at least one R group of each molecule must include a hydrolysable group such as hydroxy, alkoxy or acyloxy with hydroxy being preferred. The silicone polymer may include a further component capable of crosslinking of the general formula (MₐD_{b}T_{c}Q_{d})ₓ where M, D, T and Q are as defined above and meeting the following parameters: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group. The silicone polymer may also comprise mixtures of the polymer and the crosslinker, and may further comprise a catalyst. Preferred silicone polymers form films in the presence of moisture. Any catalyst that promotes crosslinking may be used. Preferred catalysts are metal soaps, especially preferred are tetraalkyl titanate and tetraalkyl zirconates.

The preferred silicone polymers comprise from 75 to 90% of base polymer more preferably 80 to 85%, most preferable about 82.6% and from 10 to 25% crosslinker more preferably 10 to 17% , most preferably 15% and from 1 to 5% of a catalyst preferably 2 to 3% and most preferably 2.4%. The presently preferred silicone polymer is available from GT Products, Grapevine Tex. as X5814.

The essential oil is combined with the silicone polymer. The essential oil is selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil. Preferred oils are cedar oil, cinnamon oil, citronella oil, clove oil, eucalyptus oil, juniper oil, tall oil, and pine oil. Cedar oil (also known as cedar wood oil) is especially preferred. Preferred cedar oil is available from CedarCide, Inc. of Spring, Tex.

The solid material may be any material convenient to the area where the product will be formulated and that is easily processed into a particulate material that is preferably finely divided and easily handled. Such material may include plant stems having a pithy core such as native North American marsh plants of the species hibiscus, such as H. moscheutos, H. moscheutos spp. palustris, and H. laevis (formerly H. militaris), and H. coccineous. An especially desirable plant material is corncobs ground to the composition known in the trade as “prill”. The core may also be particulate clay, expanded clay, diatomaceous earth, glass beads, silica gel beads, zeolites, soil amendments, such as gypsum or calcium sulfate particles, lime, and the like, nontoxic spent catalyst waste; fertilizer, time release fertilizer pellets, grain casings such as, rice hulls, wheat chaff and the like, nut shells such as peanut hulls, pecan shells, almond shells, walnut shells, pistachio shells, hazelnut shells, and the like, wood particles such as pelletized wood dust, sawdust, wood chips, wood shavings bark mulch, vegetative matter such as processed soy bean meal, dried vegetable wastes, pelletized vegetative matter, coir, corn cob prills, mulch, and the like.

The composition is prepared as follows: In a power stirred vessel is placed a volume of oil and the silicone-based polymer is slowly added with stirring. In this manner compositions of 80% Cedar 20% X5814; 75% Cedar oil/25% X5814; 65% Cedar oil/35% X5814 and 60% Cedar oil/and 40% X5814 are prepared. Samples of commercially available corncob prills are treated by aerosol spraying tumbled prills with a commercial paint spray gun powered by compressed air at 200 psi. Compositions containing a biologically effective amount of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil are prepared in a similar manner as that described above for cedar oil.

The invention is highly effective in treating gardens and forming a protective zone around dwellings and other buildings having landscaping substantially surrounding the structure. The exact selection of treatment compositions and materials will vary depending on the specific protection needed and the availability of treatment materials near the source of the place to be protected.

### EXAMPLE 2

Protection Against Insects

Prills comprising corncob particles were sprayed with a composition prepared as set out in example 1 and comprising 80% cedar oil 20% X5814 allowed to dry. An untreated control was also provided. The prills were placed in a test chamber and various insects collected in Spring Tex. were placed in the chamber. In each case after 24 hours, the insects were dead. Ten species of insects native to south Texas including German cockroach, cat fleas, mosquitoes, gypsy moths Japanese beetles, horned beetles, were placed in the glass aquarium and exposed to 10 grams of prills treated with a composition prepared as set out in example 1. As noted above after 24 hours, in each case the insects in the treated chamber were dead while those in the control chamber having untreated prill were active.

We claim:

1. A method of treatment useful for preventing biological pest infestation that comprises applying to an area to be protected an effective amount of a composition comprising a solid coated with a composition comprising (1) a biologically effective amount of at least one oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and the balance further comprising at least one silicone based polymer that forms a film in the presence of a catalyst and water comprising at least one component selected from the group consisting of (A) a copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; and (B) (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and (C) a crosslinking catalyst.

2. The method of claim 1 comprising an oil selected from the group consisting of cedar oil, cinnamon oil, citronella oil, clove oil, eucalyptus oil, juniper oil, tall oil, and pine oil.

3. The method of claim 1 comprising cedar oil.

4. The method of claim 1 comprising X5814

5. The method of claim 1 wherein R groups may be the same or different and each is a lower alkyl group of no more that four carbons.

6. The method of claim 1 that comprises a where all copolymer R groups are methyl.

7. The method of claim 1 that comprises a cross-linker having wherein an R group in an alkoxy group is an alkyl group each comprising from 1 to 4 carbon atoms.

8. The method of claim 7 that further comprises methyl groups at non-alkoxy position.

9. A composition of matter that comprises a solid coated with (1) a biologically effective amount of at least one oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and the balance further comprising at least one silicone based polymer that forms a film in the presence of a catalyst and water comprising at least one component selected from the group consisting of (A) a copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; and (B) (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and (C) a crosslinking catalyst.

10. The composition of claim 9 comprising an oil selected and further the oil is from the group consisting of cedar oil, cinnamon oil, citronella oil, clove oil, eucalyptus oil, juniper oil, tall oil, and pine oil.

11. The composition of claim 9 comprising an oil selected and further the oil is from the group consisting of cedar oil, cinnamon oil, citronella oil, clove oil, eucalyptus oil, juniper oil, tall oil, and pine oil.

12. The composition of claim 9 comprising cedar oil.

13. The composition of claim 9 comprising X5814

14. The composition of claim 9 wherein R groups may be the same or different and each is a lower alkyl group of no more that four carbons.

15. The composition of claim 9 that comprises a silicone where all copolymer R groups are methyl.

16. The composition of claim 9 that comprises a crosslinker having wherein an R group in an alkoxy group is an alkyl group each comprising from 1 to 4 carbon atoms.

17. The composition of claim 15 that further comprises methyl groups at non-alkoxy position.

18. The composition of claim 9 that comprises a solid selected from the group consisting of particulate clay, expanded clay, diatomaceous earth, glass beads, silica gel beads, zeolites, soil amendments, gypsum or calcium sulfate particles, lime, nontoxic spent catalyst waste; fertilizer, time release fertilizer pellets, rice hulls, wheat chaff, peanut hulls, pecan shells, almond shells, walnut shells, pistachio shells, hazelnut shells, wood particles, pelletized wood dust, sawdust, wood chips, wood shavings, bark mulch, vegetative matter such as processed soy bean meal, dried vegetable wastes, pelletized vegetative matter, coir, corn cob prills, and mulch.

19. The composition of claim 9 wherein the solid is corncob prill.

20. A method of preparing a biologically active plant protection composition comprising the steps of mixing a particulate solid material in a chamber and spraying the solid during the mixing of the solid with a mist of a composition that comprises (1) a quantity of a selected oil sufficient to provide a biologically effective amount in the finished product of at least one oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and the balance further comprising at least one silicone based polymer that forms a film in the presence of a catalyst and water comprising at least one component selected from the group consisting of (A) a copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; and (B) (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and (C) a crosslinking catalyst and curing the mixture in the presence of water to provide a free flowing particulate solid material.
